(19) United States
(12) Patent Application Publication
(10) Pub. No.: US 2025/0101487 A1
(43) Pub. Date: Mar. 27, 2025

(54) SELF-DILUTING MICROFLUIDIC DEVICE FOR RAPID ANTIMICROBIAL SUSCEPTIBILITY TESTS

(71) Applicants: The University of Hong Kong, Hong Kong (HK); Advanced Biomedical Instrumentation Centre Limited, Hong Kong (HK); Centre for Virology, Vaccinology and Therapeutics Limited, Hong Kong (HK)

(72) Inventors: Sammer Ul HASSAN, Hong Kong (HK); Ho Cheung SHUM, Hong Kong (HK); Kai-Wang Kelvin TO, Hong Kong (HK); Ka Hei WAT, Hong Kong (HK)

(21) Appl. No.: 18/472,228

(22) Filed: Sep. 22, 2023

(51) Int. Cl. C12Q 1/20; B01L 3/00

(52) U.S. Cl. CPC C12Q 1/20; B01L 3/502707; B01L 3/502715; B01L 2200/025; B01L 2200/16; B01L 2300/0819; B01L 2300/0829

(57) ABSTRACT

The present invention relates to a SDFAST (Self Dilution for Faster Antimicrobial Susceptibility Testing), which is a microfluidic device that can perform self-dilution and does not require any pumps or valves to accomplish multiplexed microfluidic processes. SDFAST is designed using AutoCAD and fabricated using micro-milling machine. It consists of two polymethyl methacrylate (PMMA) rectangular plates which are in contact throughout the operation. The first plate is the bottom one that serves as lines of wells. The second plate is the top one that acts as a lid and seals the system. The second plate has complementary designs that echo the wells in the first plate, which allows fluidic channels to be formed.

### FIELD OF THE INVENTION

The present invention generally relates to the field of microfluidics technology and microbiology, with the goal of addressing antimicrobial resistance issues and providing a more efficient method for microbial susceptibility testing.

### BACKGROUND OF THE INVENTION

Antimicrobial resistance (AMR) has been one of humanity's major health threats in the 21^{st} Century. For instance, it was estimated that AMR had contributed to 1.27 million deaths directly in 2019, with Escherichia coli (E. coli), Staphylococcus aureus (S. aureus), and Acinetobacter baumannii (A. baumannii) being a few of the most dominant pathogens¹. Numerous approaches have been devised to address this menace, with one of them involving the determination of the minimum inhibitory concentration (MIC) through antimicrobial susceptibility testing (AST).

Currently, AST is mostly commonly conducted by the broth microdilution method^{3,4}, in which the antibiotic solution of the desired concentration is prepared, injected into a microwell, and diluted serially from well to well. Afterward, an equal volume of bacteria solution is introduced to every microwell with antibiotics of varied concentrations, and the well-plate is incubated overnight. In the end, MIC is determined by observing and analyzing the turbidity of each well, and the well with the lowest concentration of antibiotic showing no turbidity represents the MIC⁵. Nonetheless, this method necessitates laboratory conditions and a lengthier duration to produce highly precise outcomes. For instance, the concordance rate of MICs determined through this approach is only 58.6% after 24 hours of incubation, and it takes 72 hours of incubation to achieve a rate exceeding 90% 6. Another common yet less complex method is disc diffusion⁷. In this technique, bacteria are initially cultured on Mueller-Hinton agar. Subsequently, antimicrobial discs are positioned on the agar plate, and the plate is left to incubate overnight. The diameters of inhibition zones, the regions where bacteria are being killed, are then measured, and these values are compared to reference standards to ascertain susceptibility. This method is used for qualitative determination of bacterial susceptibility, in which the bacteria are determined to be “resistant”, “intermediate,” or “susceptible” from the results. Despite its widespread use, this method has several drawbacks. For example, it does not assess antibiotic MICs and, therefore, cannot determine the appropriate antibiotic concentrations for prescription. Additionally, it is time-consuming, requiring 16 to 24 hours to produce results, and must be conducted in a laboratory^{8,9}. Consequently, an alternative approach that is portable, user-friendly, and time-efficient needs to be developed for rapid MIC determination.

To develop such a method, microfluidics devices are utilized. First proposed in 200910, the microfluidic device could produce repeatable experimental findings in a multiplexed environment. Depending on the research or test, it could be preloaded with reagents or user-loaded on-site. Several types of microfluidics-based platforms have been fabricated and experimented with in the past, including microchips^{11,12}, droplet-based platforms¹³⁻¹⁵, microchannels^{16,17}, dipsticks (lab-on-a-stick)¹⁸, and discs (lab-on-a-disc)¹⁹. These studies have highlighted numerous advantages of microfluidics technology compared to conventional methods, underscoring the considerable potential of these devices as point-of-care (POC) diagnostic tools^{9,20}. For example, they offer high throughput capabilities and can be readily automated to conduct testing on numerous samples, thereby minimizing errors associated with manual operation²¹. They also necessitate minimal sample and chemical volumes, thereby reducing the wastage of medical resources. Nevertheless, these devices each have their own respective disadvantages. For instance, lab-on-a-stick devices exhibit varying sensitivities and specificities, which diminish the reproducibility of results in point-of-care (POC) diagnostics²². Current droplet-based methods are not user-friendly for non-specialists, both in terms of hardware operation and software analysis¹⁵. Additionally, controlling liquid flow in microchannels and analyzing results can pose challenges for individuals who are not specialists in the field.

### SUMMARY OF THE INVENTION

To address the aforementioned drawbacks, the present invention explores a SDFAST, which is one of these microfluidic devices with straightforward functionality capable of conducting multiplexed experiments.

In a first aspect, the present invention provides a self-diluting microfluidic device for rapid antimicrobial susceptibility tests. The self-diluting microfluidic device has a top microchip and a bottom microchip, both the top microchip and the bottom microchip have nanolitre-sized wells for self-generation of dilution gradients. The bottom microchip incorporates complementary designs mirroring the wells of the top microchip, thereby creating fluidic channels connecting the ducts, and the at least two microchips remain in contact throughout entire operation. The microfluidic device combines integrated dielectrophoresis (DEP), SlipChip technology, and preloaded multiplex array PCR.

In accordance with one embodiment, the nanolitre-sized wells of the bottom microchip have a consistent size and volume, the nanolitre-sized wells of the top microchip have varying volumes, and the well volumes decreased by half for each subsequent well.

In accordance with one embodiment, the self-diluting microfluidic device is assembled through the following steps: preparing the top microchip and the bottom microchip; applying a hydrophobic layer (e.g., Aquapel™) onto a contacting surface of the top microchip and the bottom microchip; covering the contacting surface with a medium containing 1% surfactant mixture; and clipping the top microchip and the bottom microchip together to form the self-diluting microfluidic device.

In accordance with one embodiment, the medium includes FC-40.

In accordance with one embodiment, the SDFAST is a microfluidic device that does not rely on pumps or valves to carry out multiplexed microfluidic processes¹⁰.

In accordance with one embodiment, both the top microchip and the bottom microchip are fabricated through the following steps: creating a prototype of microchip by using AutoCAD software; converting the prototype of AutoCAD design into toolpaths for machining the microchip; using the toolpaths to drill holes at designated positions within the microchip to create inlets and outlets; employing the toolpaths to mill holes and connecting channels within the microchip, ensuring precision and consistency; and cutting out a product of microchip based on the design using the toolpaths.

In accordance with one embodiment, the top microchip and the bottom microchip are made from polymethyl methacrylate (PMMA).

In accordance with one embodiment, two fluidic channels are formed, a first fluidic channel of the two fluidic channel contains wells with a constant volume, while a second fluidic channel of the two fluidic channel contains wells with volumes decreasing consecutively by two-fold from left to right.

In accordance with one embodiment, the nanolitre-sized wells of the bottom microchip have dimensions of 4 mm in length, 0.5 mm in width, and 0.8 mm in height.

In accordance with another embodiment, the first four of the nanolitre-sized wells of the top microchip share the same dimensions of 4 mm in length, 0.5 mm in width, but with a decreased height in a range of 0.1 mm to 10 mm. The fifth to seventh of the nanolitre-sized wells of the top microchip share the same dimensions of 0.5 mm in width 0.1 mm in height, but with a decreased length in a range of 0.1 mm to 2 mm. The eighth well of the nanolitre-sized wells of the top microchip have dimensions of 0.5 mm in length, 0.5 mm in width and 0.05 mm in height.

In a second aspect, the present invention provides a method for expediting antimicrobial susceptibility testing, including assembling a self-diluting microfluidic device; injecting a bacteria sample with a colorimetric indicator into one or more constant-volume channels of the bottom microchip, and injecting an antibiotic solution into one or more varied-volume channels of the top microchip; slipping the top microchip so that the bottom microchip having the bacteria sample is aligned with the top microchip having the antibiotic solution, and forming one or more droplets with different sizes; combining the one or more droplets to mix the bacteria sample with the antibiotic solution; and incubating the bottom microchip and the top microchip and detecting color changes.

In accordance with another embodiment, the bacteria sample includes Escherichia coli, Acinetobacter baumannii, Klebsiella pneumoniae, and Staphylococcus species.

In accordance with another embodiment, the colorimetric indicator comprises Cell Counting Kit-8.

Compared to existing technologies, the present invention offers the following major advantages:
(1) The present invention overcomes the disadvantages of conventional methods for performing AST, such as broth microdilution, E-test, and disc diffusion, which are currently widely used in hospitals and clinics. These methods are time-consuming, taking 2-3 days to yield results. They also require specialized skills and instruments, leading to the potential wastage of medical resources.
(2) The invention requires less time for dilution, offering greater flexibility in testing. In prior literature, antibiotics had to undergo a 24-hour diffusion process before testing, meaning that the dilution process took a full day to complete. In contrast, the present invention provides a significantly more time-efficient dilution method, with on-chip dilution taking only a few seconds.
(3) The present invention leverages microfluidics and simple working principles, enabling non-professionals to easily conduct AST using nanoscale chemicals without relying on costly instruments like spectrophotometers. By incorporating a colorimetric indicator like CCK-8, the processing time is significantly reduced to 4-6 hours, making the present invention an effective point-of-care (POC) diagnostic tool.

### BRIEF DESCRIPTION OF THE DRAWINGS

Embodiments of the present invention are described in more detail hereinafter with reference to the drawings, in which:

FIG. 1 depicts a schematic diagram of comparison between SDFAST and conventional method (e.g. disc diffusion);

FIG. 2A depicts AutoCAD drawings of the top microchip. FIG. 2B depicts AutoCAD drawings of the bottom microchip. FIG. 2C depicts a varied volume of each nanowell in a line from the top microchip;

FIG. 3A depicts schematic graphs (side view) and photos that demonstrate the three phases in the mechanism of SDFAST by food dyes. FIG. 3B depicts an example of SDFAST results involves the mixing of Ciprofloxacin (CIP) with Acinetobacter baumannii (A. baumannii), utilizing the water-soluble tetrazolium salt-8 (WST-8) assay. In all three trials (represented by three lines), a significant and measurable shift in color intensity was observed, transitioning from colorless to orange;

FIG. 4 depicts leakage of solutions in SDFAST, in which the surface was pretreated with different concentrations of surfactant;

FIG. 5 depicts graph of intensity of blue food dye against volume of nanowell (A), as well as comparison of E. Coli growth in different volumes of nanowells (B).

FIG. 6 depicts chemical mechanism of CCK-8 assay.

FIG. 7A depicts changes in colour intensities in nanowell sequence from left to right in A. baumannii. FIG. 7B depicts changes in colour intensities in nanowell sequence from left to right in E. coli. FIG. 7C depicts K. pneumoniae, demonstrated by line graphs and heatmaps; and

FIG. 8 depicts line graphs and heatmaps of 16 Staphylococcus species.

### DETAILED DESCRIPTION

The present invention will be described in detail through the following embodiments with appending drawings. It should be understood that the specific embodiments are provided for an illustrative purpose only, and should not be interpreted in a limiting manner. Those skilled in the art will appreciate that the invention described herein is susceptible to variations and modifications other than those specifically described.

The invention includes all such variation and modifications. The invention also includes all of the steps and features referred to or indicated in the specification, individually or collectively, and any and all combinations or any two or more of the steps or features. Other aspects and advantages of the invention will be apparent to those skilled in the art from a review of the ensuing description.

Antimicrobial resistance (AMR) represents a worldwide public health concern that has led to numerous fatalities and the inefficient utilization of medical resources. One approach to address this issue is by conducting antimicrobial susceptibility tests (AST) on common bacteria to determine their minimum inhibitory concentrations (MIC) for the antibiotics used in treatment. Currently, these tests are predominantly conducted in clinics using methods such as broth microdilution and disc diffusion. Nonetheless, these methods have their drawbacks, including a low concordance rate, the generation of unnecessary clinical waste, and the requirement for professional execution in a laboratory.

Previous research has demonstrated the feasibility of utilizing the SDFAST in ASTs. A recent study presented the possibility of phenotypic AST using a gradient-droplet SDFAST. It generated accurate colorimetric results by mixing E. coli, including both reference strains and clinical samples from patients with urinary tract infections (UTIs), with four antibiotics, respectively²³. Meanwhile, the antibiotic solutions had to be diffused within the device for 24 hours, which was time-consuming and inflexible when testing a range of antibiotics. Another study demonstrated the applicability of the SDFAST in AST for blood samples²⁴. E. coli and S. aureus samples in blood culture were incubated with antibiotics, and the MICs or ranges were determined using entropy-based image analysis. The results proved to be more precise and accurate compared to the microdilution method, although it was less user-friendly as it necessitated complex calculations and image analysis. Although the potential of SDFAST in AST has been demonstrated, there have been only a few related studies, and the methods used in the aforementioned studies had their own respective drawbacks. Moreover, no studies have offered a more comprehensive dataset for AST using SDFAST, which includes data from mixing different species of bacteria with different antibiotics. Additionally, none have demonstrated an inlet system capable of self-diluting antibiotics.

In light of this, the present invention introduces a microfluidics device named SDFAST (Self Dilution for Faster Antimicrobial Susceptibility Testing) as a prospective point-of-care (POC) diagnostics tool and an alternative to the currently prevailing method. The microfluidics device has a top microchip and a bottom microchip, both the top microchip and the bottom microchip have nanolitre-sized wells for self-generation of dilution gradients. The bottom microchip incorporates complementary designs mirroring the wells of the top microchip, thereby creating fluidic channels connecting the ducts, and the at least two microchips remain in contact throughout entire operation.

In accordance with one embodiment, the self-diluting microfluidic device is assembled through the following steps: preparing the top microchip and the bottom microchip; applying a hydrophobic layer (e.g., Aquapel™) onto a contacting surface of the top microchip and the bottom microchip; covering the contacting surface with a medium (e.g., FC-40) containing 1% surfactant mixture; and clipping the top microchip and the bottom microchip together to form the self-diluting microfluidic device. After applying hydrophobic layer and fluorosurfactant to the chip surfaces, the chips are clipped together, creating fluidic channels. Two types of channels are formed: one set contains wells with a constant and identical volume, while the other set consists of wells with volumes decreasing consecutively by two-fold from left to right.

In accordance with one embodiment, both the top microchip and the bottom microchip are fabricated through the following steps: creating a prototype of microchip by using AutoCAD software; converting the prototype of AutoCAD design into toolpaths for machining the microchip; using the toolpaths to drill holes at designated positions within the microchip to create inlets and outlets; employing the toolpaths to mill holes and connecting channels within the microchip, ensuring precision and consistency; and cutting out a product of microchip based on the design using the toolpaths.

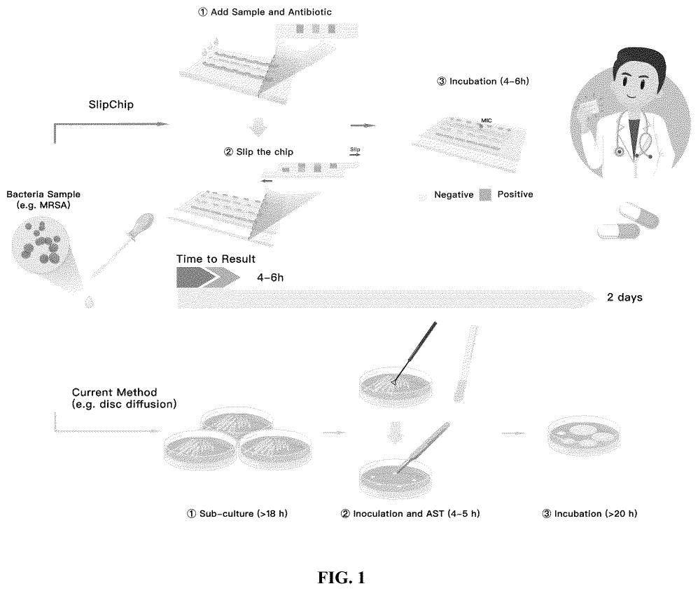

In the present invention, the design of SDFAST is capable of self-performing serial dilutions and user-friendly, particularly for non-professionals. FIG. 1 depicts a schematic diagram of comparison between SDFAST and conventional method. The present invention incorporates the function of self-dilution, an unprecedented feature in SDFAST research and design. Although studies on SDFAST AST have been conducted, demonstrating its effectiveness and accuracy, they did not incorporate self-dilution in their design. In those studies, antibiotic solutions had to be diluted outside of the system before reacting with bacteria, which was time-consuming. In contrast, the present invention allows for in-system dilution: the solution to be diluted is injected into the line of wells with varying volumes. Subsequently, the upper chip is slid upwards, and droplets with different volumes, representing different concentrations, can be formed within seconds. This design not only significantly reduces the dilution time but also conserves materials.

Additionally, conventional methods need to be carried out in laboratory settings and demand specific training. For example, in broth microdilution, solutions must be meticulously diluted using pipettes, and the wells can be prone to contamination, necessitating several months of practice for trainees to achieve proficiency and avoid errors. Furthermore, these methods produce results that may either require expensive instruments for analysis or be challenging to interpret. For instance, broth microdilution necessitates the use of a spectrophotometer, and E-test MIC results can occasionally be misinterpreted since they rely on visual observation of bacterial growth. However, the present invention overcomes these demerits by introducing device with simple mechanism and experimental procedures-after simple pretreatment with Aquapel™ and surfactant, testers only need to assemble the chips, inject solutions, slip the chips, and incubate the device. Combining with cell viability kit such as CCK-8 or resazurin, the MIC can be located easily by observing the obvious colorimetric changes in wells. The present invention enables non-professionals to conduct AST and easily and quickly observe its results.

In one of the embodiments, the microfluidics device prevented leakage and contamination of samples by adding surfactant, such that the solution in wells remains droplets.

In one of the embodiments, only two lines of wells were required. The dilution was done by slipping and forming the droplets with different sizes, and the mixing was done by combining the droplets together. These two procedures could be completed in just a few seconds.

In one of the embodiments, the microfluidics device was tested by mixing 16 strains of Staphylococcus samples with Vancomycin (VAN) under the colorimetric WST-8 assay, as well as demonstrated its usage in different scenarios by testing A. baumannii, E. coli and Klebsiella pneumoniae (K. pneumoniae) with Ceftazidime (CTZ), Ciprofloxacin (CIP) and Levofloxacin (LEV).

Additionally, the present invention also provides a method for expediting antimicrobial susceptibility testing, including assembling a self-diluting microfluidic device; injecting a bacteria sample with a colorimetric indicator into one or more constant-volume channels of the bottom microchip, and injecting an antibiotic solution into one or more varied-volume channels of the top microchip; slipping the top microchip so that the bottom microchip having the bacteria sample is aligned with the top microchip having the antibiotic solution, and forming one or more droplets with different sizes; combining the one or more droplets to mix the bacteria sample with the antibiotic solution; and incubating the bottom microchip and the top microchip and detecting color changes.

In current procedures, patients can only receive prescriptions after waiting several days for AST results. This can inconvenience patients and worsen their health conditions. By combining the invention with a rapid colorimetric method, AST results can be obtained quickly, enabling patients to receive prescriptions on the same day they visit the clinic or hospital for testing samples. Furthermore, the concept of self-dilution in microfluidic devices holds high potential and offers diverse applications. The invention can be applied not only in antimicrobial testing but also in other research fields that require testing samples at variable concentrations, such as glucose level monitoring or biomarker testing.

The following examples illustrate the present invention and are not intended to limit the same.

### EXAMPLE

### Example 1—Fabrication of SDFAST

Polymethyl Methacrylate (PMMA) serves as the material choice for manufacturing SDFAST due to its outstanding optical characteristics, appropriate hardness combined with scratch resistance, along with its superior impact strength and dimensional stability. Out of all the various commercially available plastics, PMMA possesses the highest surface hardness²⁵.

The fabrication process was done by a 2.5D micro milling machine LPKF ProtoMat S104²⁶. Because of its increased speed and flexibility, the milling process was selected over the current chemical etching methods. Moreover, the time taken for the fabrication process of each SDFAST was short, under 30 minutes, and the parameters could be easily modified with the system application on the computer.

The tools used in the fabrication process of SDFAST were divided into five steps: (1) prototyping using AutoCAD; (2) conversion into respective toolpaths; (3) drilling of inlets and outlets; (4) milling of wells and connecting channels; and (5) cutting out the final product at last.

In one embodiment, the SDFAST was designed using CAD software (SolidWorks, Dassault Systemes), and nanowells were micromachined on a polymethyl methacrylate (PMMA) piece (3 mm thickness) using a 2.5D micro milling machine LPKF ProtoMat S104.

In the first step of prototyping, AutoCAD was employed as the design tool. The design was solely derived from the top view, as micro-milling applications only accepted 2.5D objects in the past. After confirming the parameters and design at the software, the file was converted into dxf. file format and imported into the micro milling software. In the second step, the software accepted the design and converted it into a CAM diagram within the user interface. Several operations were done to generate the required tool paths. The different cutting layers were assigned into a 2.5D milling layer and a 2.5D drilling layer, respectively. For example, the nanowells and connecting channels were designated to the 2.5D milling layer, while the inlets and outlets were designated to the 2.5D drilling layer.

The cutting areas were subsequently transformed into polygons to make them compatible with the machine as 3D objects. The height of each component was then specified using the 2.5D blind function. The 2.5D cutting function was then employed to create the toolpath. Appropriate tools were chosen to ensure the patterns could be accurately fabricated. The fabrication process commenced with the machine referencing, during which the height of the material was measured using the pressure sensor located at the drill's head. The machine proceeded to calibrate the distance between the material surface and the tool tip. The drilling toolpaths were given priority in the process. A 0.9 mm spiral drilling tool was chosen to create the inlets and outlets on the chip. Following this, the fabrication process continued with the milling toolpaths. The 0.5 mm End Mill tool was used.

In the current process, all the nanowells and connecting channels were produced. Once all the channels within the chip were finished, the chip was separated from the PMMA sheet using 1 mm/2 mm End Mill tools. Upon the completion of all the toolpaths, the chip was removed from the machine using a pair of clean clips. The chip's surface was initially cleansed using compressed air to eliminate dirt and excess debris. Subsequently, it was rinsed with deionized water and ethanol. Afterward, the chips underwent another round of cleaning with compressed air to facilitate drying. Finally, the plates were sealed in clean Petri dishes for storage to prevent contamination.

### Example 2—Design of SDFAST

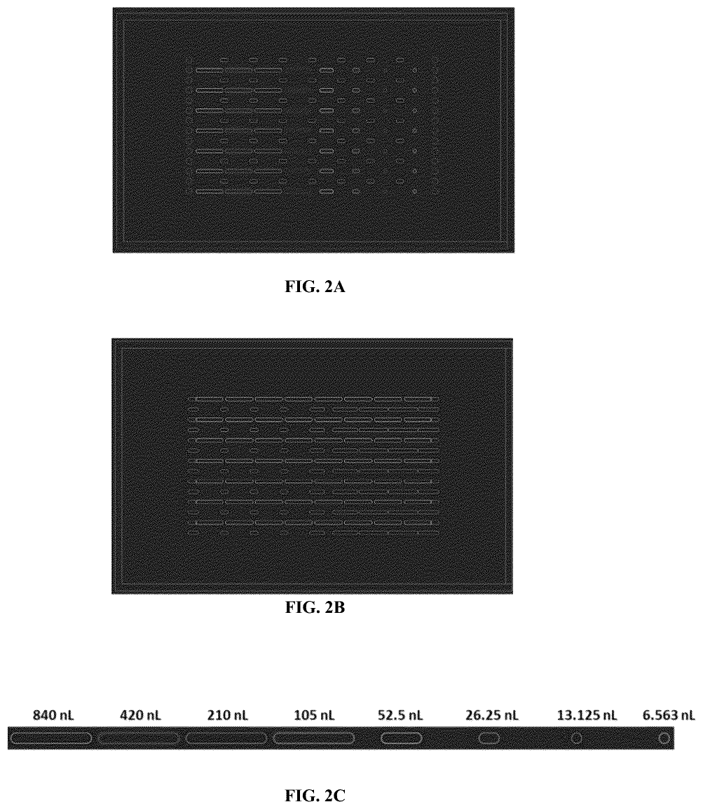

The SDFAST designed for testing AMR comprised a top chip (FIG. 2A) and a bottom chip (FIG. 2B). Both chips were manufactured from 3 mm PMMA using the micro-milling machine. The unique feature of this chip was the ability to perform serial dilution with a simple mechanism. The bottom chip had a consistent size and volume. In contrast, the top chip featured nanowells with varying volumes. The well volumes decreased by half for each subsequent well. For example, the second well had a volume half that of the first well. The rows of constant and variable wells were arranged in alternating patterns. The chip had a total of 12 rows of wells, enabling it to conduct six trials of serial dilution mixing simultaneously.

Regarding the wells with constant volume, the parameters of the wells were 4 mm (length)×0.5 mm (width)×0.8 mm (height). Referring to FIG. 2C, in the case of the wells with variable volume, the first four wells shared the same dimensions of 4 mm (length)×0.5 mm (width) but with a decreased height. The first well had a height of 0.8 mm. The second well had a height of 0.4 mm, the third well had a height of 0.2 mm, and the fourth well had a height of 0.1 mm. The fifth, sixth, and seventh wells had the same height of 0.1 mm but decreased length, and they had lengths of 2 mm, 1 mm, and 0.5 mm, respectively. The eighth well had the parameter of 0.5 mm (length)×0.5 mm (width) and 0.05 mm (height). All the wells were interconnected with 0.5 mm deep connecting channels and included an inlet and an outlet. A microfluidic channel was formed within each row of these channels.

### Example 3—Assembly of the SDFAST

First, the chips were rinsed with deionized water and dried. Then, a thin layer of Aquapel™ was applied to the contacting surfaces of the two acrylic plates. This treatment rendered the chip surface hydrophobic. The chips were then placed into an oven for 15 minutes at 60° C. for the Aquapel™ to bond to the surface. Afterward, a 1% surfactant mixture in FC-40 was prepared and applied to the contacting surfaces to ensure complete coverage. The FC-40 facilitated sealing and had a high boiling point of 158-173° C., which prevented the issue of evaporation¹⁰. This ensured the controlled volume of sample and reactants to generate more accurate and reproducible experimental results. After treatment, the plates were clipped together and ready for use.

In one of the embodiments, a surfactant was added to FC-40 to increase its surface tension, preventing the droplet from adhering to the chip surface during the slipping process.

### Example 4—Mechanism of SDFAST

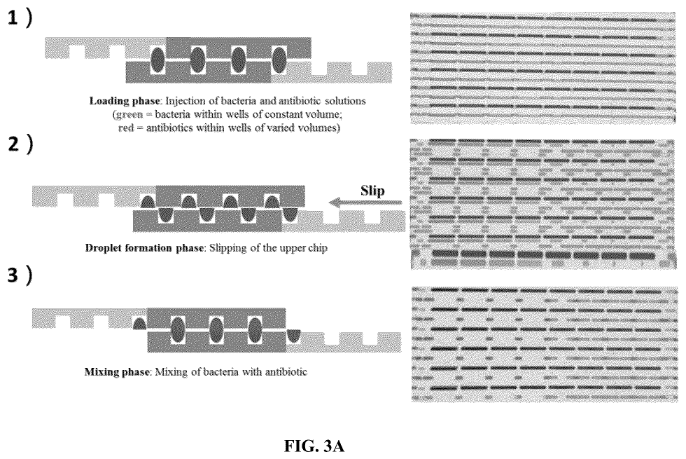

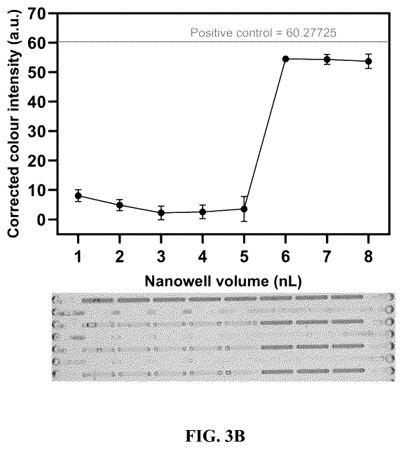

SDFAST (Self Dilution for Faster Antimicrobial Susceptibility Testing) involved a relatively simple mechanism with three phases: loading, droplet formation, and mixing (FIG. 3A). In the loading phase, a bacteria sample with a colorimetric indicator (CCK-8, cell counting kit-8) was injected into the constant-volume channels, while the antibiotic solution is injected into the varied-volume channels. In the droplet formation phase, the upper chip was slipped, forming droplets. In the mixing phase, the bacteria sample was mixed with the antibiotic solution. The chips were then incubated and were observed for any color changes (for CCK-8, it required 4-6 hours of incubation, and the color changed from colorless to bright orange). The well before the abrupt color change represented the minimum inhibitory concentration (MIC) of the bacteria with the antibiotic.

During the loading phase, the inlets and wells were aligned, creating a fluidic path along each line of wells. The reactants were then introduced into the wells through the inlets, completely filling all the wells, typically using a 10 μL pipette tip. The tip was inserted into the inlet, ensuring a precise fit to prevent any leakage during fluid loading. The micropipette button was gently pressed, and the fluid was loaded slowly into the wells. The filling process continued until the outlet was completely filled. The outlet remained open during the loading phase to facilitate pressure dissipation.

After that, the chips were pushed into the droplet formation phase, resulting in the creation of independent droplets. The inlets and outlets were disconnected from the fluidic channels, creating a closed system that encapsulated the droplets within the chip. The top chip was then advanced to the second step in the mixing phase, where the reactants and samples in the corresponding wells overlapped, allowing for mixing. In this phase, chemical reactions could occur, and changes in color intensity or fluorescence signals could be measured. For instance, differences in color intensity between wells with varying antibiotic concentrations could be measured (as shown in FIG. 3B). It should be noted that the first and last rows of wells on the chip should not be included in the experimental data since they only served as connecting channels, and no reagent mixing or chemical reactions took place in those rows.

### Example 5—Optimization and Characterization of SDFAST

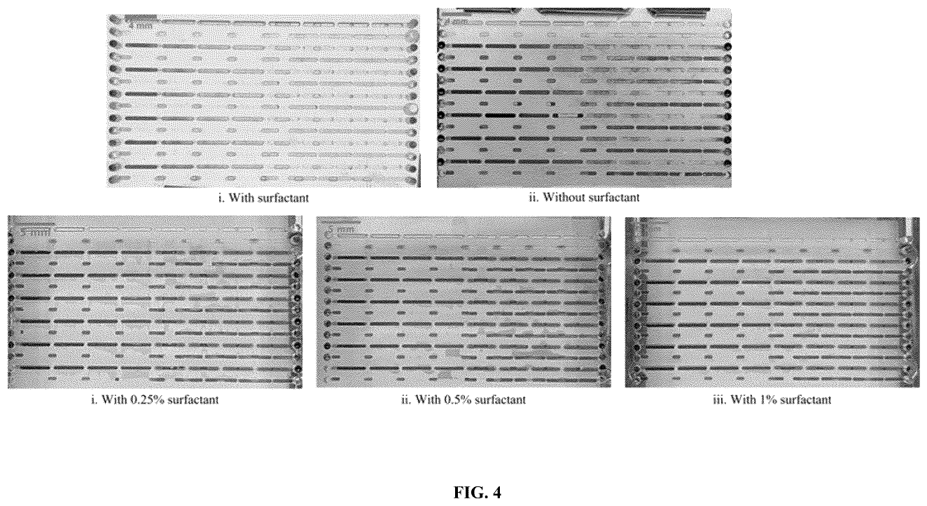

Before experimenting with bacteria and antibiotics, optimization and characterization of SDFAST were carried out to maximize its efficiency. Testing with crystal violet showed that applying surfactant in FC-40 on SDFAST was necessary to prevent leakage. Meanwhile, the optimal concentration of surfactant in FC-40 had to be determined, as leakage occurred when no or too little surfactant was added. At the same time, the solutions in wells could not mix well when the surfactant concentration was too high. A food dye test concluded that 1% was the optimal concentration as the most negligible leakage was observed (FIG. 4).

The injections were divided into three sets, with the first two serving as control lines and the last one as the testing line: (1) Two positive control lines were filled with a bacterial solution (5% WST-8); (2) two negative control lines were filled with phosphate-buffered saline (PBS); and (3) one line had a constant volume in each well, filled with a bacterial solution (5% WST-8), while the other line had varying volumes and was filled with an antibiotic solution.

The solutions were injected using a micropipette with 0.1-20 μL tips. For lines with constant volume, 20 μL solution was required to fill up one entire line, while for lines with varied volumes, 10 μL solution was required. The excess solution was expelled from the hole adjacent to the end side of each line and could be wiped with KimWipes. After filling the wells, the chips were slid so that the lower line aligned and overlapped with the upper line, allowing the solutions to mix. Subsequently, the chips were placed inside a Petri dish containing FC-40, and the dish was sealed with parafilm. The dish was then incubated at 37° C. for 6 hours before color analysis using ImageJ.

The SDFAST's capability to perform serial dilution was based on the relationship between nanowell volume and the concentration of the injected solution. The SDFAST was first tested with blue dye, and the result showed a logarithmic relationship between the colour intensity and nanowell volume. As the volume of nanowell decreased from left to right, the colour intensity also decreased.

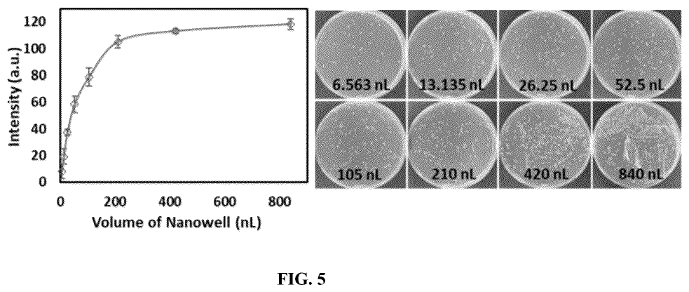

Another test with E. coli also showed the same trend, in which the number of colonies reduced as the nanowell volume decreased. Both results demonstrated that serial dilution could be performed in the device itself (FIG. 5).

### Example 6—Choice of Colorimetric Method

A variety of colorimetric methods are available for the qualification and quantification of bacteria, including resazurin¹⁸, the iodine-starch test with glucose and glucose oxidase²⁷, p-Benzoquinone²⁸, and water-soluble tetrazolium salt-8 (WST-8; also known as Cell Counting Kit-8 or abbreviated as CCK-8). Eventually, the WST-8 assay was chosen as it has been proven to be an accurate and quick colorimetric method to determine MIC^{6,29,30}.

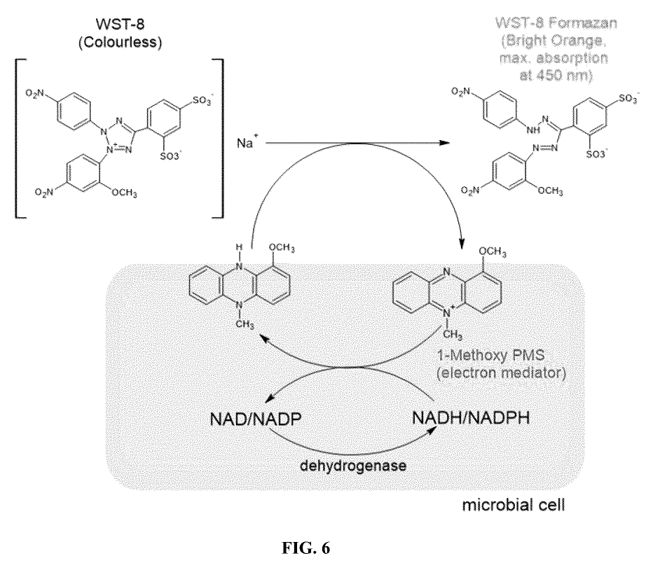

Results could be obtained after 6 hours of incubation and were minimally affected by external factors, such as acid and non-cellular reduction of WST-8³¹. Moreover, the color change along the line of wells is distinct and sudden, making it easy to determine the MIC from the results, and only a minimal volume of indicator (10 μL) is needed for each trial. In the assay, the resulting WST-8 formazan produces a bright orange color, with the maximum absorption occurring at 450 nm (for the chemical mechanism of WST-8, see FIG. 6)³².

### Example 7-Efficacy Test

One of the main applications of SDFAST is in drug resistance study. This example described how to perform antimicrobial susceptibility test (AST) with SDFAST.

To prove the accuracy of SDFAST AST under several conditions, three species of bacteria, namely Escherichia coli (E. Coli), Acinetobacter baumannii (A. baumannii), and Klebsiella pneumoniae (K. pneumoniae) were selected, and three antibiotics, namely Ceftazidime (CTZ), Ciprofloxacin (CIP), and Levofloxacin (LVX), were selected for the experiment.

The bacteria were cultured on an agar plate and incubated in Mueller-Hinton Broth (MHB, Sigma-Aldrich) at 37° C. overnight. The culture solution was then diluted 1000 times with MHB and mixed with 5% WST-8 indicator (Beyotime). The antibiotic solutions were prepared by dissolving the desired amount of antibiotics (solid or liquid) into DI water, and the solutions were diluted by DI water according to the desired concentrations to be tested. CIP, which was only slightly soluble in water, was dissolved by adding a few drops of 37% hydrochloric acid (HCl) beforehand.

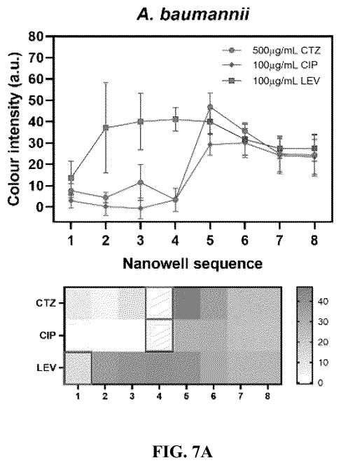

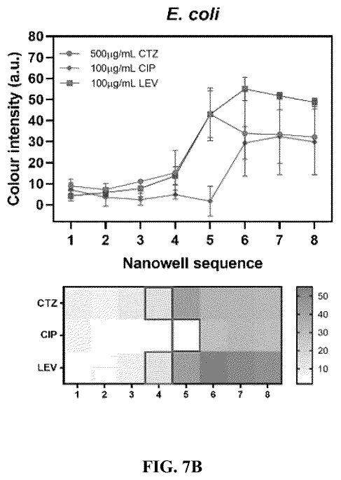

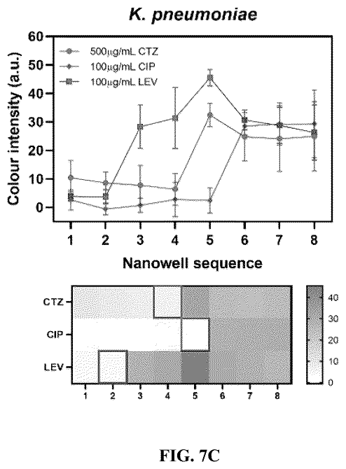

In the present invention, the SDFAST was operated with the water-soluble tetrazolium salt-8 (WST-8) assay, in which four prevalent bacteria, Acinetobacter baumannii (A. baumannii), Escherichia coli (E. coli), Klebsiella pneumoniae (K. pneumoniae) and clinical samples containing Staphylococcus species were tested under various antibiotics. Referring to FIGS. 7A-7C, the quantification of WST-8 results on A. baumannii, E. coli, and K. pneumoniae showed that the colour intensity increased quickly as the antibiotic concentration reached below the corresponding MIC. All the bacteria-antibiotic combinations showed a general trend, in which the colour intensity was low for the first few wells, but rised abruptly at a particular well and then remained higher than the first few wells as the sequence shifted to the right. The colourless or very pale yellow well before the abrupt change in each line represented the corresponding MIC, which could be easily identified by the naked eyes. After the identification, the MIC value was calculated based on dilution rates.

Table 1 showed a comparison between the MIC values obtained from SDFAST and broth microdilution. The comparison between the values from SDFAST and those from broth microdilution showed that their MICs were similar or only one well apart from each other, which indicated the accuracy of the device of the present invention and that it could be an alternative to the conventional method, regardless of the different genera of bacteria as well as types of antibiotics.

TABLE 1

| | | | Broth microdilution |
|---|---|---|---|
| Bacteria | Antibiotics | SDFAST (μg/mL) | (μg/mL) |
| A. baumannii | CTZ | 62.5 | 62.5-125 |
| | CIP | 6.25-12.5 | 25 |
| | LEV | 100 | 100 |
| E. coli | CTZ | 62.5 | 62.5 |
| | CIP | 6.25 | 6.25-12.5 |
| | LEV | 12.5 | 12.5 |
| K. pneumoniae | CTZ | 62.5 | 125 |
| | CIP | 6.25 | 12.5 |
| | LEV | 50 | 50 |

The above results demonstrated that SDFAST was an effective method, providing clear results even for users with limited knowledge in microbiology and analytical chemistry. They could readily identify the well representing the MIC by observing the abrupt color change in the testing line, allowing them to calculate the MIC based on the dilution rate.

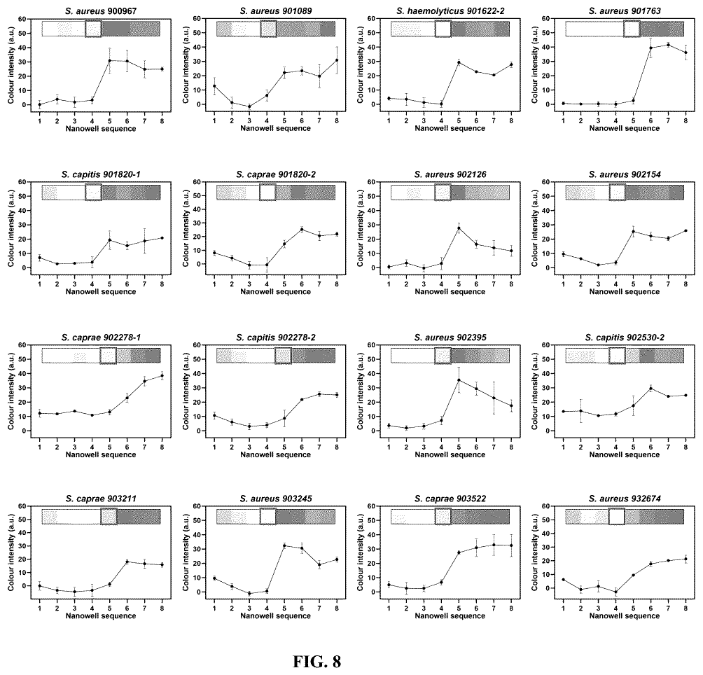

To further verify the accuracy of SDFAST and demonstrate the performance of the device of the present invention with a wide variety of species in one genus, 16 strains of Staphylococcus species (aureus, capitis, caprae and haemolyticus) were tested and compared with another conventional method E-test. They were obtained from the Department of Microbiology, Queen Mary Hospital, Hong Kong (Patient samples). Vancomycin hydrochloride solution (100 mg/mL in DMSO, Sigma-Aldrich) was diluted with DI water to desired concentrations for each strain. The results were compared to those obtained by E-test from the Department of Microbiology. The results were similar to the previous section, in which the same trend was observed in all the samples, and the wells representing MICs could be easily identified (FIG. 8).

Table 2 also showed a comparison between the MIC values obtained from SDFAST and E-test. The comparison revealed the similarity in results between SDFAST and the references. In the present invention, SDFAST could accurately identify the MICs in a short period of time. Compared to broth microdilution and E-test, which necessitate overnight incubation, the microfluidics device of the present invention could determine the MICs within just 6 hours of incubation. In some cases, this duration could be further reduced to 4 hours.

TABLE 2

| | | E-test (reference value) |
|---|---|---|
| MRSA Strain | SDFAST (μg/mL) | (μg/mL) |
| S. aureus 900967 | 1 | 1 |
| S. aureus 901089 | 1 | 1 |
| S. haemolyticus 901622-2 | 1.56 | 1.5 |
| S. aureus 901763 | 0.5 | 0.5 |
| S. capitis 901820-1 | 1 | 1 |
| S. caprae 901820-2 | 1.56 | 1.5 |
| S. aureus 902126 | 0.77-1.4 | 0.75 |
| S. aureus 902154 | 0.94 | 0.5 |
| S. caprae 902278-1 | 0.195 | 0.19 |
| S. capitis 902278-2 | 1 | 1 |
| S. aureus 902395 | 0.5 | 0.5 |
| S. capitis 902530-2 | 0.55 | 0.5 |
| S. caprae 903211 | 0.5 | 0.5 |
| S. aureus 903245 | 1.4 | 0.75 |
| S. caprae 903522 | 1.11 | 1 |
| S. aureus 932674 | 0.77-1.4 | 0.75 |

### Example 8—Quantification of Data

The efficiency of SDFAST was assessed based on the color intensity of WST-8. To quantify color intensity, photos of the chip were captured with a Canon camera and analyzed using ImageJ's ‘Measure’ function. The first obtained value was the positive control value after subtracting the negative control (background). This ensured that the sample could be analyzed independently of the background and served as a reference for comparison with values from the testing line. The “mean” of each well in the positive line was first measured, and the raw positive control value was calculated by averaging all the “means”. The same measuring and averaging procedures were done for negative control. Finally, the positive control value was obtained by deducting the raw by negative control. The “mean” of each well was measured to analyze the testing line. The negative control was deducted by each “mean” value to yield a series of corrected color intensity (y-axis values).

At last, a graph of corrected color intensity along the nanowell sequence (from the 1^{st} well to the 8^{th} well), as well as a heatmap of colour intensity, was plotted using GraphPad Prism 8. In the line graph, the error was represented by the standard deviation (S.D.) value from averaging the colour intensities from three trials.

### Example 9—Comparison with Existing Methods

The most novel aspect of the present invention was the self-dilution feature. This was accomplished by creating wells with varying sizes on the microchip surface, while keeping the volume of diluent fixed on the chip. This eliminated the requirement of additional steps for dilution generations and hence allowed injection into wells in a single slipping step. Previous research and designs have primarily emphasized the interaction between samples in different wells during slipping, specifically the mixing phase, but they did not incorporate features aimed at streamlining the manual and repetitive procedures preceding the main reaction, such as the preparation of different solution concentrations. It was believed that including these features was also crucial, as potential users could have been non-professionals or had limited access to medical resources. These features reduced the need for training non-professionals in performing preliminary procedures manually, as well as the need for medical resources.

Some prior literatures utilized entropy analysis to find out MIC values, which required both costly devices and professionals to perform. For instance, an inverted microscope was required to take pictures at every 30 minutes interval to capture the activity of bacteria. The calculation was also complicated, with the analysis of the pixels of images that requires professional knowledge. In contrast, the present invention utilized colorimetric analysis which could be performed just by naked eye observation and simple calculation of dilution factors, which was cheaper, more user-friendly and simpler to be operated. In addition, the present invention also removed the need to preload the antibiotics with varied concentrations, as the system itself could generate them.

Although both designs in the prior literature and the present invention demonstrated the self-dilution property, the mechanisms of dilution were different. For example, the design from the prior literature utilized gradient diffusion to dilute the solution. In that approach, a dilution buffer was injected into a line of interconnected wells, while the concentrated solution to be diluted was added to a stock chamber. When the chips were slipped to bring the two solutions into contact, dilution began through diffusion. In the present invention, dilution was achieved by injecting the solution to be diluted into a line of wells with varying volumes. When the chips were slipped, droplets with different volumes were formed, completing the dilution process. Both designs demonstrated the on-chip dilution function; however, their mechanisms of dilution differed. In previous research, the solution to be diluted was injected into a line of wells with the same volume, while the dilution medium was injected into an adjacent line of wells with varying volumes. By slipping the chips, the solution and the dilution medium could be combined to generate a chain of diluted droplets. However, in the present invention, the solution to be diluted was injected into a line of wells with different volumes. As a result, once the chips were slipped, the diluted droplets were immediately formed. The design of the present invention included only two types of lines of wells (those with constant and varied volumes, respectively), making it suitable for fields like point-of-care diagnosis.

In summary, the SDFAST of the present invention is a simple and effective microfluidic device that functions as a point-of-care (POC) diagnostics tool. The device has two microchips, which allow the injection of samples with bacteria and antibiotics by forming fluidic paths after connecting the chips. The solutions can be mixed by sliding one microchip against another with a single press of the microchip.

The results have demonstrated the advantages of the device of the present invention. It not only allows for the simple and quicker determination of MIC by non-professionals without the need for a spectrophotometer but also operates with significantly fewer medical resources compared to conventional methods, such as E-test and disc diffusion. The experiments have shown that SDFAST effectively facilitates the mixing of bacteria and antibiotics, providing readings that can be easily comprehended and analyzed, even by users with limited knowledge of relevant subjects.

### Definitions

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

“MIC”, defined as the minimum concentration of an antibiotic that inhibits the visible growth of a specific type of bacteria after overnight incubation², represents a critical indicator of drug resistance. The accurate determination of MICs for antibiotics in a short timeframe can help mitigate antimicrobial resistance resulting from antibiotic overuse. For instance, healthcare providers can prescribe antibiotics at appropriate concentrations to patients, thereby preventing overuse.

“Aquapel™” is a type of glass surface treatment, commonly used to enhance the water repellency and rain resistance of car windshields. It can improve the flow of rainwater on the glass surface, reduce adhesion of raindrops, making water beads more likely to roll off the glass, thereby improving driving visibility. This coating is typically applied to the front windshield of vehicles to enhance driving safety. The application of Aquapel™ can improve the driving experience in rainy and wet conditions.

“FC-40 medium” is a type of fluorinated carbon medium, commonly used as a liquid medium in microfluidic devices and laboratory applications. It is a fluorinated carbon oil characterized by its low surface tension and chemical inertness, which provides certain advantages in microfluidic applications, such as reducing the interfacial tension between droplets, helping to maintain droplet integrity, and minimizing interactions between fluids like oil and water. FC-40 is typically employed in microfluidic experiments, especially in droplet-based microfluidics and droplet biology applications, to aid in controlling and manipulating tiny droplets and reagents.

CCK-8 (Cell Counting Kit-8) is a commonly used assay kit in biological and cellular research. It is a colorimetric indicator used to assess cell proliferation and cellular metabolic activity. The principle of CCK-8 is based on the relationship between cell viability and the metabolic products of cells. When cells are in an active state, they reduce a chemical compound present in CCK-8 to produce a colored product. This colored product can be measured for its absorbance through spectrophotometry or colorimetric analysis, allowing researchers to determine cell quantity and metabolic activity. CCK-8 reagent is employed for rapid and sensitive detection of cellular responses to substances such as drugs, toxins, or other factors that may affect cell growth.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

It will be appreciated by those skilled in the art, in view of these teachings, that alternative embodiments may be implemented without undue experimentation or deviation from the spirit or scope of the invention, as set forth in the appended claims. This invention is to be limited only by the following claims, which include all such embodiments and modifications when viewed in conjunction with the above specification and accompanying drawings.

### INDUSTRIAL APPLICABILITY

The market of antimicrobial susceptibility testing (AST) has been growing and becoming more competitive. In 2022, manual and conventional AST (especially disc diffusion methods) has dominated the market. In the field of microfluidics technology, medical applications hold the largest market share at 83.8%. Within this technology type, lab-on-a-chip represents the largest sector, accounting for 37.83% of the global market. These showed that utilizing lab-on-a-chip in medical application has a great potential.

Market research on AST technology indicates that the high cost of AST automation is a significant barrier in the market. The present invention addresses this challenge by eliminating the need for automation and the associated costs. The use of standard laboratory equipment such as ovens and incubators, which are fundamental instruments in biological or clinical labs, eliminates the necessity to purchase additional automated instruments specifically for on-chip AST. By integrating AST with on-chip microfluidic technology, this invention is poised to address common challenges in the AST market while capitalizing on the advantages offered by lab-on-a-chip technology in a relatively new domain.

References: The disclosures of the following references are incorporated by reference
[1] C. J. L. Murray et al., “Global burden of bacterial antimicrobial resistance in 2019: a systematic analysis,” Lancet, vol. 399, no. 10325, pp. 629-655, 2019.
[2] J. M. Andrews, “Determination of minimum inhibitory concentrations,” Journal of Antimicrobial Chemotherapy, vol. 49, no. 6, pp. 5-16, 2001.
[3] N. Rezaei, Ed., Encyclopedia of Infection and Immunity, Elsevier, 2022.
[4] C. R. Belanger and R. E. W. Hancock, “Testing physiologically relevant conditions in minimal inhibitory concentration assays,” Nature Protocols, vol. 16, pp. 3761-3774, 2021.
[5] M. Balouiri, M. Sadiki and S. K. Ibnsouda, “Methods for in vitro evaluating antimicrobial activity: A review,” Journal of Pharmaceutical Analysis, vol. 6, no. 2, pp. 71-79, 2016.
[6] T. Tsukatani et al., “Comparison of the WST-8 colorimetric method and the CLSI broth microdilution method for susceptibility testing against drug-resistant bacteria,” Journal of Microbiological Methods, vol. 90, no. 3, pp. 160-166, 2012.
[7] H. J, “Kirby-Bauer Disk Diffusion Susceptibility Test Protocol,” American Society for Microbiology, 2009. [Online]. Available: https://asm.org/getattachment/2594ce26-bd44-47f6-8287-0657aa9185ad/Kirby-Bauer-Disk-DiffusionSusceptibility-Test-Protocol-pdf.pdf. [Accessed 10 Mar. 2023].
[8] R. M. Humphries et al., “The Continued Value of Disk Diffusion for Assessing Antimicrobial Susceptibility in Clinical Laboratories: Report from the Clinical and Laboratory Standards Institute Methods Development and Standardization Working Group,” Journal of Clinical Microbiology, vol. 56, no. 8, 2018.
[9] M. Benkova, O. Soukup and J. Marek, “Antimicrobial susceptibility testing: currently used methods and devices and the near future in clinical practice,” Journal of Applied Microbiology, vol. 129, no. 4, pp. 806-822, 2020.
[10] W. Du, L. Li, K. P. Nicholsa and R. F. Ismagilov, “SlipChip,” Lab on a Chip, vol. 9, no. 16, p. 2286-2292, 2009.
[11] S. C. Kim, S. Cestellos-Blanco, K. Inoue and R. N. Zare, “Miniaturized Antimicrobial Susceptibility Test by Combining Concentration Gradient Generation and Rapid Cell Culturing,” Antibiotics, vol. 4, no. 4, pp. 455-466, 2015.
[12] M. Safavieh et al., “Rapid Real-Time Antimicrobial Susceptibility Testing with Electrical Sensing on Plastic Microchips with Printed Electrodes,” ACS Applied Materials & Interfaces, vol. 9, no. 14, pp. 12832-12840, 2017.
[13] A. M. Kaushik et al., “Accelerating bacterial growth detection and antimicrobial susceptibility assessment in integrated picoliter droplet platform,” Biosensors and Bioelectronics, vol. 97, pp. 260-266, 2017.
[14] W. Postek and P. Garstecki, “Droplet Microfluidics for High-Throughput Analysis of Antibiotic Susceptibility in Bacterial Cells and Populations,” Accounts of Chemical Research, vol. 55, no. 5, pp. 605-615, 2022.
[15] A. Ruszczak, S. Bartkova, M. Zapotoczna, O. Scheler and P. Garstecki, “Droplet-based methods for tackling antimicrobial resistance,” Current Opinion in Biotechnology, vol. 76, no. 102755, 2022.
[16] Y. Matsumoto et al., “A Microfluidic Channel Method for Rapid Drug-Susceptibility Testing of Pseudomonas aeruginosa,” PLOS One, vol. 11, no. 2, 2016.
[17] C. H. Chen et al., “Rapid Antimicrobial Susceptibility Testing Using High Surface-to-Volume Ratio Microchannels,” Analytical Chemistry, vol. 82, no. 3, pp. 1012-1019, 2010.
[18] N. M. Reis, J. Pivetal, A. L. Loo-Zazueta, J. M. S. Barrosb and A. D. Edwards, “Lab on a stick: multi-analyte cellular assays in a microfluidic dipstick,” Lab on a Chip, vol. 16, no. 15, p. 2891-2899, 2016.
[19] V. Sunkara, S. Kumar, J. Sabaté Del Río, I. Kim and Y. K. Cho, “Lab-on-a-Disc for Point-of-Care Infection Diagnostics,” Accounts of Chemical Research, vol. 54, no. 19, pp. 3643-3655, 2021.
[20] S. Hassan and X. Zhang, “Microfluidics as an Emerging Platform for Tackling Antimicrobial Resistance (AMR): A Review,” Current Analytical Chemistry, vol. 16, no. 1, pp. 41-51, 2020.
[21] X. Zhu, J. Chu and Y. Wang, “Advances in Microfluidics Applied to Single Cell Operation,” Biotechnology Journal, vol. 13, no. 1700416, 2018.
[22] J. Hu et al., “Advances in paper-based point-of-care diagnostics,” Biosensors and Bioelectronics, vol. 54, pp. 585-597, 2014.
[23] X. Liu et al., “Formation and Parallel Manipulation of Gradient Droplets on a Self-Partitioning SlipChip for Phenotypic Antimicrobial Susceptibility Testing,” ACS Sensors, vol. 7, no. 7, pp. 1777-2101, 2022.
[24] Q. Yi et al., “Direct antimicrobial susceptibility testing of bloodstream infection on SlipChip,” Biosensors and Bioelectronics, vol. 135, pp. 200-207, 2019.
[25] A. Hashim and B. Abbas, “Recent Review on Poly-methyl methacrylate (PMMA)-Polystyrene (PS) Blend Doped with Nanoparticles For Modern Applications,” Research Journal of Agriculture and Biological Sciences, vol. 14, no. 3, pp. 6-12, 2019.
[26] S. Hassan, A. M. Nightingale and X. Niu, “Micromachined optical flow cell for sensitive measurement of droplets in tubing,” Biomedical Microdevices, vol. 20, no. 92, 2018.
[27] J. Sun, J. Huang, Y. Li, J. Lv and X. Ding, “A simple and rapid colorimetric bacteria detection method based on bacterial inhibition of glucose oxidase-catalyzed reaction,” Talanta, vol. 197, pp. 304-309, 2019.
[28] J. Sun, A. R. Warden, J. Huang, W. Wang and X. Ding, “Colorimetric and Electrochemical Detection of Escherichia coli and Antibiotic Resistance Based on a p-Benzoquinone-Mediated Bioassay,” Analytical Chemistry, vol. 91, no. 12, pp. 7524-7530, 2019.
[29] M. Y. Jung et al., “Applicability of a colorimetric method for evaluation of lactic acid bacteria with probiotic properties,” Food Microbiology, vol. 64, pp. 33-38, 2017.
[30] X. Yang, Y. Zhong, D. Wang and Z. Lu, “A simple colorimetric method for viable bacteria detection based on cell counting Kit-8,” Analytical Methods, vol. 13, pp. 5211-5215, 2021.
[31] T. Tsukatani et al., “Colorimetric microbial viability assay based on reduction of water-soluble tetrazolium salts for antimicrobial susceptibility testing and screening of antimicrobial substances,” Analytical Biochemistry, vol. 393, no. 1, pp. 117-125, 2009.
[32] K. Chamchoy, D. Pakotiprapha, P. Pumirat, U. Leartsakulpanich and U. Boonyuen, “Application of WST-8 based colorimetric NAD (P) H detection for quantitative dehydrogenase assays,” BMC Biochemistry, vol. 20, no. 4, 2019.

What is claimed is:

1. A self-diluting microfluidic device for rapid antimicrobial susceptibility tests, wherein the self-diluting microfluidic device comprises a top microchip and a bottom microchip, both the top microchip and the bottom microchip have nanolitre-sized wells for self-generation of dilution gradients, the bottom microchip incorporates complementary designs mirroring the wells of the top microchip, thereby creating fluidic channels connecting the ducts, and wherein the at least two microchips remain in contact throughout entire operation, and the nanolitre-sized wells of the bottom microchip have a consistent size and volume, the nanolitre-sized wells of the top microchip have varying volumes, and well volumes decreased by half for each subsequent well.

2. The self-diluting microfluidic device of claim 1, wherein the self-diluting microfluidic device is assembled through the following steps: preparing the top microchip and the bottom microchip; applying a hydrophobic layer onto a contacting surface of the top microchip and the bottom microchip; covering the contacting surface with a medium containing 1% surfactant mixture; and clipping the top microchip and the bottom microchip together to form the self-diluting microfluidic device.

3. The self-diluting microfluidic device of claim 2, wherein the medium comprises FC-40.

4. The self-diluting microfluidic device of claim 1, wherein both the top microchip and the bottom microchip are fabricated through the following steps:
creating a prototype of microchip by using AutoCAD software;
converting the prototype of AutoCAD design into toolpaths for machining the microchip;
using the toolpaths to drill holes at designated positions within the microchip to create inlets and outlets;
employing the toolpaths to mill holes and connecting channels within the microchip, ensuring precision and consistency; and
cutting out a product of microchip based on the design using the toolpaths.

5. The self-diluting microfluidic device of claim 1, wherein the self-diluting microfluidic device uses no pumps or valves to accomplish multiplexed microfluidic processes.

6. The self-diluting microfluidic device of claim 1, wherein the top microchip and the bottom microchip are made from polymethyl methacrylate (PMMA).

7. The self-diluting microfluidic device of claim 1, wherein two fluidic channels are formed, a first fluidic channel of the two fluidic channel contains wells with a constant volume, while a second fluidic channel of the two fluidic channel contains wells with volumes decreasing consecutively by two-fold from left to right.

8. The self-diluting microfluidic device of claim 1, wherein the nanolitre-sized wells of the bottom microchip have dimensions of 4 mm in length, 0.5 mm in width, and 0.8 mm in height.

9. The self-diluting microfluidic device of claim 1, wherein the first four of the nanolitre-sized wells of the top microchip share the same dimensions of 4 mm in length, 0.5 mm in width, but with a decreased height in a range of 0.1 mm to 10 mm.

10. The self-diluting microfluidic device of claim 9, wherein the fifth to seventh of the nanolitre-sized wells of the top microchip share the same dimensions of 0.5 mm in width 0.1 mm in height, but with a decreased length in a range of 0.1 mm to 2 mm.

11. The self-diluting microfluidic device of claim 10, wherein the eighth well of the nanolitre-sized wells of the top microchip have dimensions of 0.5 mm in length, 0.5 mm in width and 0.05 mm in height.

12. A method for expediting antimicrobial susceptibility testing, comprising:
assembling a self-diluting microfluidic device of claim 1;
injecting a bacteria sample with a colorimetric indicator into one or more constant-volume channels of the bottom microchip, and injecting an antibiotic solution into one or more varied-volume channels of the top microchip;
slipping the top microchip so that the bottom microchip having the bacteria sample is aligned with the top microchip having the antibiotic solution, and forming one or more droplets with different sizes;
combining the one or more droplets to mix the bacteria sample with the antibiotic solution; and
incubating the bottom microchip and the top microchip and detecting color changes.

13. The method of claim 12, wherein the self-diluting microfluidic device is assembled through the following steps: preparing the top microchip and the bottom microchip; applying a hydrophobic layer onto a contacting surface of the top microchip and the bottom microchip; covering the contacting surface with a medium containing 1% surfactant mixture; and clipping the top microchip and the bottom microchip together to form the self-diluting microfluidic device.

14. The method of claim 13, wherein the self-diluting microfluidic device uses no pumps or valves to accomplish multiplexed microfluidic processes.

15. The method of claim 12, wherein the bacteria sample comprises Escherichia coli, Acinetobacter baumannii, Klebsiella pneumoniae, and Staphylococcus species.

16. The method of claim 12, wherein the colorimetric indicator comprises Cell Counting Kit-8.

17. The method of claim 12, wherein both the top microchip and the bottom microchip are fabricated through the following steps:
creating a prototype of microchip by using AutoCAD software;
converting the prototype of AutoCAD design into toolpaths for machining the microchip;
using the toolpaths to drill holes at designated positions within the microchip to create inlets and outlets;
employing the toolpaths to mill holes and connecting channels within the microchip, ensuring precision and consistency; and
cutting out a product of microchip based on the design using the toolpaths.
